(19) United States
(12) Patent Application Publication
(10) Pub. No.: US 2017/0035701 A1
(43) Pub. Date: Feb. 9, 2017

(54) STABILIZED HIGH DRUG LOAD NANOCARRIERS, METHODS FOR THEIR PREPARATION AND USE THEREOF

(71) Applicant: TAIPEI MEDICAL UNIVERSITY, TAIPEI CITY (TW)

(72) Inventors: MING-THAU SHEU, TAIPEI CITY (TW); HSIU-O HO, TAIPEI CITY (TW); SHING CHUAN SHEN, TAIPEI (TW); YUAN SOON HO, TAIPEI CITY (TW); JUN-JEN LIU, TAIPEI (TW)

(21) Appl. No.: 14/817,618

(22) Filed: Aug. 4, 2015

(51) Int. Cl. A61K 9/51; A61K 31/7048; A61K 9/107; A61K 31/12; A61K 31/05; A61K 31/353; A61K 31/337; A61K 31/4745

(52) U.S. Cl. CPC A61K 9/5123; A61K 31/337; A61K 31/7048; A61K 31/4745; A61K 31/12; A61K 31/05; A61K 31/353; A61K 9/1075; A61K 9/5192; A61K 9/5146

(57) ABSTRACT

The present invention relates to generally to pharmaceutical formulations. Particularly, the present invention relates to a drug nanocarrier that is stabilized by lipids, preferably lecithins and/or lipid-terminated polyalkylene glycol, for the delivery of poorly soluble drugs with high drug loading and its utility in the fields of pharmaceutical formulation, drug delivery, medicine and diagnosis.

### FIELD OF THE INVENTION

The present invention relates generally to pharmaceutical formulations. Particularly, the present invention relates to a drug nanocarrier that is stabilized by lipids, preferably lecithins and/or lipid-terminated polyalkylene glycol, for the delivery of poorly soluble drugs with high drug loading and its utility in the fields of pharmaceutical formulation, drug delivery, medicine and diagnosis.

### BACKGROUND OF THE INVENTION

The formulation and administration of water-insoluble or sparingly water-soluble drugs, such as docetaxel, are problematic in general because of the difficulty of achieving sufficient systemic bioavailability. Low aqueous solubility results not only in decreased bioavailability, but also in formulations that are insufficiently stable over extended storage periods. For the most part, research has focused on entrapment of drugs in vesicles or liposomes, and on the incorporation of surfactants into their formulations.

Representative liposomal drug delivery systems are described in U.S. Pat. Nos. 5,395,619, 5,340,588, and 5,154,930. Liposomes, as is well known in the art, are vesicles comprised of concentrically ordered lipid bilayers that encapsulate an aqueous phase. Liposomes form when phospholipids (amphipathic compounds having a polar (hydrophilic) head group covalently bound to a long-chain aliphatic (hydrophobic) tail) are exposed to water. That is, in an aqueous medium, phospholipids aggregate to form a structure in which the long-chain aliphatic tails are sequestered within the interior of a shell formed by the polar head groups. Unfortunately, use of liposomes for delivering many drugs has proven unsatisfactory, in part because liposome compositions are, as a general rule, rapidly cleared from the bloodstream. Finally, even if satisfactory liposomal formulations could be prepared, it might still be necessary to use some sort of physical release mechanism so that the vesicle releases the drugs in the body before the liver and spleen take up the agent.

Micelles have also been used for drug delivery, as exemplified by the disclosure in U.S. Pat. No. 5,736,156 regarding camptothecin. Micelles are defined as spherical receptacles comprised of a single monolayer defining a closed compartment. Generally, amphipathic molecules such as surfactants and fatty acids spontaneously form micellar structures in polar solvents. In contrast to liposome bilayers, micelles are “sided” in that they project a hydrophilic, polar outer surface and a hydrophobic interior. Since they are monolayers, they are extremely limited in size, seldom exceeding 30 nanometers in diameter. This limited size reduces their effective encapsulation potential as drug carriers.

Among other notable drug delivery formulations, nanocrystals of drugs or carrier-stabilized drugs have been described in the art (for example, in U.S. Pat. No. 5,399,363 to Liversidge et al.). Liversidge et al. describe the production of nanoparticles of hydrophobic drugs, including natural camptothecin, using surfactants and grinding. They mention a number of surfactants, including poloxamers, and list lecithin as a stabilizing material, but provide no disclosure of types of lipids or specific formulations containing polymers and lipids. Also, while the formulations disclosed by Liversidge et al. provide a way for maximizing drug delivery capacity, their crystalline nature is problematic because of the well-known phenomenon of crystal growth over time. To overcome crystal growth, nanoparticulate crystals are sometimes coated with crystal growth-inhibiting agents such as nonionic surfactants. In these instances, care must be exerted to insure biocompatibility and nontoxicity of the surfactant or other coating agent. US Patent Application 2003/0059465 by Unger et al. revealed lipid-stabilized nanoparticles of camptothecin and camptothecin analogs (SN-38). The drug is complexed with a stabilizing agent, but is not covalently bound thereto. Anionic or neutral lipids and/or polymers are used as the stabilizing agent, and secondary stabilizing agents and/or other excipients may be incorporated into the formulation as well. However, the drug loading in stabilized nanoparticles with a size range appropriate for intravenous injection is usually less than 5%.

Another way to improve drug delivery is to formulate medications into nanoparticles. By so doing, for example, hydrophobic or toxic drugs can be more safely delivered. The nanoparticles used for such purposes should be as small as possible, preferably less than 100 nanometers in diameter. US Patent Application Publication (US2010/0203142) by ZHANG et al. provides a controlled-release system, comprising a plurality of target specific stealth nanoparticles, wherein the nanoparticles comprise a polymeric matrix, an amphipilic layer within or outside of the polymeric matrix for stability, targeting moieties covalently attached to the outer surface of the nanoparticle, and a therapeutic agent. However, the preparations exemplified illustrate that the drug loading of this lipid-stabilized PLGA nanoparticles is less than 5%.

While many nanocarrier systems suitable for intravenous administration of water-insoluble or sparingly water-soluble drugs, such as docetaxel, can be stabilized by an amphiphilic component, in particular lecithin, it still is difficult to create one that simultaneously possesses high encapsulating efficiency and high drug loading to accommodate sufficient amounts of drug. It is desirable to identify nanoparticle formulations and preparation conditions that can encapsulate sufficient drug with higher drug loading while still retaining optimal stability and physical characteristics.

### SUMMARY OF THE INVENTION

An aspect of the present disclosure is to provide a nanocarrier, comprising a lipid shell enclosing a micellar core encapsulating an active agent or a diagnostic agent, wherein the lipid shell comprises one or more amphiphilic lipids, and the micellar core comprises one or more amphiphilic polymers wherein the core optionally comprises an emulsifier.

According to one embodiment of the present disclosure, the diameter of the nanocarrier is in the range of about 50 nm to about 500 nm, preferably about 100 nm to about 500 nm, more preferably about 110 nm to about 200 nm or about 120 nm to about 150 nm. In another embodiment, the nanocarrier of the invention has an encapsulating efficiency in the range of about 50 to about 100%, preferably about 80 to about 95%, more preferably about 90 nm to about 95%.

According to some embodiments of the present disclosure, the amphiphilic lipid is selected from the group consisting of lipid-polyethyleneglycol conjugate, phospholipid, or cholesterol or a combination thereof. Examples of the phospholipid include, but are not limited to, lecithin, soybean lecithin, egg yolk lecithin, a synthetic phospholipid or a pegylated phospholipid. Preferably, the phospholipid is soybean lecithin. Examples of the synthetic phospholipid include but are not limited to phosphatidylcholine, phosphatidic acid, phosphatidylethanolamine, phosphatidylglycerol, phosphatidylserine, phosphatidylinositol, or a combination thereof. In a further embodiment, the amphiphilic lipid is a lipid-polyethyleneglycol conjugate, pegylated phospholipid, or a combination thereof. In one embodiment of the present disclosure, the lipid shell comprises a phospholipid and another amphiphilic lipid selected from pegylated phospholipid and cholesterol.

According to further embodiments of the present disclosure, the lipid shell comprises a lipid-polyethyleneglycol conjugate, pegylated phospholipid, or a combination thereof. In another embodiment, the lipid shell comprises a phospholipid and a pegylated phospholipid or cholesterol.

According to some embodiments of the present disclosure, the amphiphilic polymer is selected from the group consisting of phospholipid, poloxamer, poloxamine, L121, TPGS, tween, or ethoxylated hydrogenated castor oil, pegylated phospholipid, PLGA, PLA, PGA, and a combination thereof. Preferably, the amphiphilic polymer is phospholipid, Pluronic P123, DSPE-PEG2000, L121, TPGS or PLGA or a combination thereof. Examples of the phospholipid are as described herein. Preferably, the phospholipid is lecithin or soybean lecithin. Examples of the emulsifier include but are not limited to sodium glycocholate, sodium taurocholate and sodium taurodeoxycholate. Preferably, the emulsifier is sodium glycocholate.

According to some further embodiments of the present disclosure, the micellar core comprises a phospholipid and another amphiphilic polymer selected from DSPE-PEG2000, PLGA, Pluronic P123 and a combination thereof. In another further embodiment, the micellar core comprises a combination of lecithin and Pluronic P123 or a combination of lecithin and sodium glycolate.

According to some embodiments of the present disclosure, the active agent is pharmaceutically or nutraceutically acceptable. According to a further embodiment of the present disclosure, the active agent is a hydrophobic agent. According to some further embodiments of the present disclosure, the active agent is an anti-cancer drug, an antimicrobial drug or a nutraceutical agent.

According to some embodiments of the present disclosure, the diagnostic agent is pharmaceutically acceptable. For example, diagnostic agents may be selected from the group consisting of an imaging agent, a contrasting agent, an enzyme, a fluorescent substance, a luminescent substance and a paramagnetic molecule.

Another aspect of the present disclosure is to provide a method of preparing a nanocarrier with higher bioactive or diagnostic agent loading, comprising (i) preparing a nanosuspension comprising one or more amphiphilic lipids by subjecting the one or more amphiphilic lipids to ultrasonication; (iia) preparing a thin film comprising a mixture of one or more amphiphilic polymers wherein the mixture optionally contains an emulsifier and an active agent or a diagnostic agent by dissolving the mixture in an organic solvent and then removing the organic solvent or (iib) dissolving the mixture in an organic solvent to form an organic solution; (iii) hydrating the thin film of (iia) with the nanosuspension or injecting the organic solution of (iib) into the nanosuspension to form a solution containing self-assembling micelles encapsulating the active agent or diagnostic agent; and (iv) subjecting the micellar solution to ultrasonication at a temperature lower than 50° C. until the amphiphilic lipid forms a lipid shell and then encloses micelles as a core.

According to one embodiment of the present disclosure, the method further comprises a step of removing water from the nanocarrier aqueous solution to obtain nanocarrier in powder form. Preferably, the water is removed by freeze-drying.

According to one embodiment of the present disclosure, cup ultrasonication is utilized in the method at full power for 5 min while maintaining the temperature of solution at 25° C.

According to one embodiment of the present disclosure, the nanosuspension contains an amphiphilic lipid having a weight ratio (w/w) to an active agent or a diagnostic agent of about 1.0 to 5.0, preferably about 2.0 to 3.0, prepared at a concentration of 1.0-5.0% (w/v).

According to one embodiment of the present disclosure, the amount of the amphiphilic polymer in the thin film or in the organic solution is at a weight ratio (w/w) to active ingredient of about 1.0-10, preferably about 2.5-5.0. In another embodiment, the organic solvent is ethanol.

### BRIEF DESCRIPTION OF THE DRAWINGS

FIG. 1A and 1B are schematic diagrams of a drug carrier according to one embodiment of this disclosure.

FIG. 2 is a flow diagram of a method for preparing a nanocarrier according to one embodiment of this disclosure.

FIG. 3 shows transmission electron microscopic images of three docetaxel nanocarriers according to one embodiment of this disclosure.

FIG. 4 is a drug release rate graph of three nanocarriers at pH 7.4 environments according to one embodiment of this disclosure.

FIG. 5 is the volume change of tumor xerograph illustrated by a mouse model treated with drugs.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the examples and the sequence of steps for constructing and operating the examples. However, the same or equivalent functions and sequences may be accomplished by different examples.

As used herein, the singular forms “a” “an” and “the” include plural referents unless the context clearly dictates otherwise. Therefore, reference to, for example, a micelle includes aspects having two or more such micelles, unless the context clearly indicates otherwise.

The use of the alternative (e.g., “or”) herein should be understood to mean either one, both, or any combination thereof of the alternatives.

The term “and/or” as used herein should be understood to mean either one, both, or any combination thereof. By way of example, “A and/or B” includes “A” or “B” or “A and B.”

As described herein, any concentration range, percentage range, ratio range or integer range is to be understood to include the value of any integer within the recited range and, when appropriate, fractions thereof (such as one tenth and one hundredth of an integer), unless otherwise indicated.

As used herein, “about” means +/−15% of the indicated value or range, unless otherwise indicated.

As used herein, the term “nanocarrier” refers to a carrier with a nanosized structure in which at least one of its phases has one or more dimensions (length, width or thickness) in the nanometer size range.

As used herein, the term “patient” or “subject” refers to an animal, generally a mammal, especially including a domesticated animal and preferably a human, to whom treatment, including prophylactic treatment (prophylaxis), with the compounds or compositions according to the present invention is provided.

As used herein, the term “effective” refers to an amount of a compound or component which, when used within the context of its use, produces or effects an intended result, whether that result relates to the prophylaxis and/or therapy of an infection and/or disease state or as otherwise described herein. The term “effective” subsumes all other effective amount or effective concentration terms (including the term “therapeutically effective”) which are otherwise described or used in the present application.

As used herein, the terms “treat,” “treating,” and “treatment” are used synonymously to refer to any action providing a benefit to a patient at risk for or afflicted with a disease, including improvement in the condition through lessening, inhibition, suppression or elimination of at least one symptom, delay in progression of the disease, prevention, delay in or inhibition of the likelihood of the onset of the disease, etc.

The term “pharmaceutically acceptable” as used herein denotes that the compound or composition is suitable for administration to a subject, including a human patient, to achieve the treatments described herein, without unduly deleterious side effects in light of the severity of the disease and necessity of the treatment.

The term “active agent” refers to any biologically active compound or drug which may be formulated for use in an embodiment of the present invention. Exemplary bioactive agents include the compounds according to the present invention which are used to treat cancer or a disease state or condition which occurs secondary to cancer and may include antiviral agents as well as other compounds or agents which are otherwise described herein.

The term “diagnostic” or “diagnostic agent” used herein is any chemical moiety that may be used for diagnosis or imaging a patient.

### Nanocarrier of the Invention

In one aspect, the present invention provides a nanocarrier comprising a lipid shell enclosing a micellar core encapsulating an active agent or a diagnostic agent, wherein the lipid shell comprises one or more amphiphilic lipids, and the micellar core comprises one or more amphiphilic polymers wherein the core optionally comprises an emulsifier.

The nanocarrier is a core-shell nano-structure particle. The lipid shell encloses the micellar core and preferably the micellar core is uniformly dispersed within the lipid shell. In one embodiment, the diameter of the nanocarrier is in the range of about 50 nm to about 500 nm, preferably about 100 nm to about 500 nm, more preferably about 110 nm to about 200 nm or about 120 nm to about 150 nm. In another embodiment, the nanocarrier of the invention has an encapsulating efficiency in the range of about 50 to about 100%, preferably about 80 to about 95%, more preferably about 90 nm to about 95%.

The lipid shell of the nanocarrier of the invention comprises one or more amphiphilic lipids. Examples of the amphiphilic lipid include, but are not limited to, lipid-polyethyleneglycol conjugate, phospholipid, or cholesterol or a combination thereof. Examples of the phospholipid include, but are not limited to, lecithin, soybean lecithin, egg yolk lecithin, a synthetic phospholipid or a pegylated phospholipid. Preferably, the phospholipid is soybean lecithin. Examples of the synthetic phospholipid include, but are not limited to phosphatidylcholine, phosphatidic acid, phosphatidylethanolamine, phosphatidylglycerol, phosphatidylserine, phosphatidylinositol, or a combination thereof. In a further embodiment, the amphiphilic lipid is a lipid-polyethyleneglycol conjugate, pegylated phospholipid, or a combination thereof. In one embodiment of the present disclosure, the lipid shell comprises a phospholipid and another amphiphilic lipid selected from pegylated phospholipid and cholesterol.

In a further embodiment, the lipid shell comprises a lipid-polyethyleneglycol conjugates, pegylated phospholipid, or a combination thereof. In another embodiment, the lipid shell comprises a phospholipid and a pegylated phospholipid or cholesterol.

The micellar core of the nanocarrier of the invention comprises one or more amphiphilic polymers wherein the core optionally comprises an emulsifier. Examples of the amphiphilic polymer include, but are not limited to, phospholipid, poloxamer, poloxamine, L121, TPGS, tween, or ethoxylated hydrogenated castor oil, pegylated phospholipid, PLGA, PLA, PGA, and a combination thereof. Preferably, the amphiphilic polymer is phospholipid, Pluronic P123, DSPE-PEG2000, L121, TPGS or PLGA or a combination thereof. Examples of the phospholipid are as described herein. Preferably, the phospholipid is lecithin or soybean lecithin. Examples of the emulsifier include, but are not limited to, sodium glycocholate, sodium taurocholate and sodium taurodeoxycholate. Preferably, the emulsifier is sodium glycocholate.

In a further embodiment, the micellar core comprises a phospholipid and another amphiphilic polymer selected from DSPE-PEG2000, PLGA, Pluronic P123 and a combination thereof. In another further embodiment, the micellar core comprises a combination of lecithin and Pluronic P123 or a combination of lecithin and sodium glycolate.

According to one embodiment of the present disclosure, the above-mentioned active agent is pharmaceutically or nutraceutically acceptable. According to a further embodiment of the present disclosure, the active agent is a hydrophobic agent. According to another further embodiment of the present disclosure, the active agent is an anti-cancer drug, an antimicrobial drug or a nutraceutical agent. Examples of the active agent include, but are not limited to, docetaxel, paclitaxel, irinotecan, SN-38, amphotericin B, curcumin, resveratrol, quercetin, honokiol or magnolol.

According to another embodiment of the present disclosure, the above-mentioned diagnostic agent is a pharmaceutically acceptable. For example, diagnostic agents include, but are not limited to, imaging agents containing radioisotopes such as indium or technetium; contrasting agents containing iodine, technetium, or gadolinium; enzymes such as horse radish peroxidase, GFP, alkaline phosphatase, or beta-galactosidase; fluorescent substances such as fluorescein, rhodamine and europium derivatives; luminescent substances such as N-methylacrydium derivatives or the like and paramagnetic molecules.

According to yet another embodiment of the present disclosure, the loading of the active agent in the nanocarrier is in the range of about 5% to about 15%.

In another aspect, the present invention provides a method of preparing a nanocarrier with higher bioactive or diagnostic agent loading, comprising (i) preparing a nanosuspension comprising one or more amphiphilic lipids by subjecting the one or more amphiphilic lipids to ultrasonication; (iia) preparing a thin film comprising a mixture of one or more amphiphilic polymers wherein the mixture optionally comprises an emulsifier and an active agent or a diagnostic agent by dissolving the mixture in an organic solvent and then removing the organic solvent or (iib) dissolving the mixture in an organic solvent to form an organic solution; (iii) hydrating the thin film of (iia) with the nanosuspension or injecting the organic solution of (iib) into the nanosuspension to form a solution containing self-assembling micelles encapsulating the active agent or diagnostic agent; and (iv) subjecting the micellar solution to ultrasonication at a temperature of lower than 50° C. until the amphiphilic lipid forms a lipid shell and then encloses micelles as a core.

In one embodiment, the first step is to prepare a phospholipid nanosuspension by subjecting phospholipid dispersion to ultrasonication and a thin film or an ethanol solution. Self-assembling micelles encapsulating a pharmaceutically or nutraceutically active ingredient are produced by hydrating the thin film composed of an amphiphilic polymer and a pharmaceutically or nutraceutically active ingredient with phospholipid nanosuspension or by injection of ethanol solution containing an amphiphilic polymer and a pharmaceutically or nutraceutically active ingredient into this phospholipid nanosuspension. The next step is to subject this micellar solution to ultrasonication at a temperature of lower than 50° C. until a lipid shell encloses a micellar core composed of an amphiphilic polymer, wherein a pharmaceutically or nutraceutically active ingredient is disposed.

According to an embodiment of the present disclosure, the phospholipid nanosuspension contains lecithin having a weight ratio (w/w) to active ingredient about 1.0 to 5.0, preferably about 2.0 to 3.0, prepared at a concentration of 1.0-5.0% (w/v).

According to another embodiment of the present disclosure, the phospholipid nanosuspension further contains cholesterol having a weight ratio (w/w) to lecithin about 0.0 to 0.5, preferably about 0.1 to 0.2.

According to another embodiment of the present disclosure, the used amount of the amphiphilic polymer in the thin film or in ethanol solution is at a weight ratio (w/w) to active ingredient about 1.0-10, preferably about 2.5-5.0.

According to yet another embodiment of the present disclosure, the amphiphilic polymer is TPGS, L121, or DSPE-PEG2000, and a combination of them with a used amount being at a weight ratio (w/w) to active ingredient about 1.0-10, preferably about 2.5-5.0.

According to yet another embodiment of the present disclosure, the method for ultrasonication is using an ultrasonic processor (VCX 750-750 Watts; Frequency: 20 kHz, Sonics and Materials, Inc.) and the temperature is preferably 25° C.

According to yet another embodiment of the present disclosure, the method further comprises a step of removing water from nanocarrier aqueous solution containing nanocarrier to obtain nanocarrier in powder form.

It is to be understood that both the foregoing general description and the following detailed description are examples, and are intended to provide further explanation of the disclosure as claimed.

An aspect of the present disclosure is to provide a nanocarrier composed of a lipid shell enclosing micelles, which are dispersed uniformly within the lipid shell. The lipid shell comprises a phospholipid with or without lipid; the micelles comprise an amphiphilic polymer with or without phospholipid or emulsifier and the micelles enclose a pharmaceutically or nutraceutically active ingredient.

According to another embodiment of the present disclosure, the phospholipid is lecithin, soybean lecithin, egg yolk lecithin or a synthetic phospholipid. The synthetic phospholipid is selected from phosphatidylcholines, phosphatidic acid, phosphatidylethanolamines, phosphatidylglycerols, phosphatidylserines, phosphatidylinositols, and a combination thereof. Lipid is amphiphilic lipid or cholesterol.

According to another embodiment of the present disclosure, the above-mentioned amphiphilic lipid is selected from lipid-polyethyleneglycol conjugates, pegylated phospholipid, and a combination thereof.

According to yet another embodiment of the present disclosure, the above-mentioned amphiphilic polymer is selected from poloxamers, poloxamines, TPGS, tween, or ethoxylated hydrogenated castor oil, pegylated phospholipid, PLGA, PLA, PGA, and a combination thereof. Emulsifier is sodium glycocholate, sodium taurocholate, or sodium taurodeoxycholate.

According to another embodiment of the present disclosure, pharmaceutically or nutraceutically active ingredients include but are not limited to docetaxel, paclitaxel, irinotecan, SN-38, amphotericin B, curcumin, resveratrol, quercetin, honokiol and magnolol.

According to yet another embodiment of the present disclosure, the diameter of the drug nanocarrier is in the range of about 50 nm to about 500 nm.

According to yet another embodiment of the present disclosure, the drug loading in the nanocarrier is in the range of about 5% to about 15%.

### Preparation of Drug Nanocarrier of the Invention

In another aspect, the invention provides a of preparing a nanocarrier of the invention with higher bioactive or diagnostic agent loading, comprising (i) preparing a nanosuspension comprising one or more amphiphilic lipids by subjecting the one or more amphiphilic lipids to ultrasonication; (iia) preparing a thin film comprising a mixture of one or more amphiphilic polymers wherein the mixture optionally comprises an emulsifier and an active agent or a diagnostic agent by dissolving the mixture in an organic solvent and then removing the organic solvent or (iib) dissolving the mixture in an organic solvent to form an organic solution; (iii) hydrating the thin film of (iia) with the nanosuspension or injecting the organic solution of (iib) into the nanosuspension to form a solution containing self-assembling micelles encapsulating the active agent or diagnostic agent; and (iv) subjecting the micellar solution to ultrasonication at a temperature of lower than 50° C. until the amphiphilic lipid forms a lipid shell and then encloses micelles as a core.

In one embodiment, the method further comprises a step of removing water from the nanocarrier aqueous solution to obtain nanocarrier in powder form. Preferably, the water is removed by freeze-drying.

In one embodiment, cup ultrasonication is utilized in the method at full power for 5 min while maintaining the temperature of solution at 25° C.

In one embodiment, the nanosuspension contains an amphiphilic lipid having a weight ratio (w/w) to an active agent or a diagnostic agent of about 1.0 to 5.0, preferably about 2.0 to 3.0, prepared at a concentration of 1.0-5.0% (w/v).

In another embodiment, the amount of the amphiphilic polymer in the thin film or in the organic solution is at a weight ratio (w/w) to active ingredient of about 1.0-10, preferably about 2.5-5.0. In another embodiment, the organic solvent is ethanol.

In a further embodiment, the amphiphilic polymer is TPGS, L121, DSPE-PEG2000, or a combination thereof. In another further embodiment, the weight ratio of the amphiphilic polymer to the active agent or diagnostic agent is about 1.0-10, preferably about 2.5-5.0.

In one embodiment, the first step of preparing the nanocarrier of the invention is to prepare a phospholipid nanosuspension by subjecting phospholipid dispersion to ultrasonication and a thin film or an ethanol solution comprising a mixture of one or more amphiphilic polymers wherein the mixture optionally comprises an emulsifier and an active agent or a diagnostic agent. Self-assembling micelles encapsulating a pharmaceutically or nutraceutically active ingredient are produced by hydrating the thin film composed of an amphiphilic polymer and a pharmaceutically or nutraceutically active ingredient with phospholipid nanosuspension or by injection of ethanol solution containing an amphiphilic polymer and a pharmaceutically or nutraceutically active ingredient into this phospholipid nanosuspension. The next step is to subject this micellar solution to ultrasonication at a temperature of lower than 50° C. until a lipid shell enclosing a micellar core composed of an amphiphilic polymer is formed, wherein a pharmaceutically or nutraceutically active ingredient is disposed.

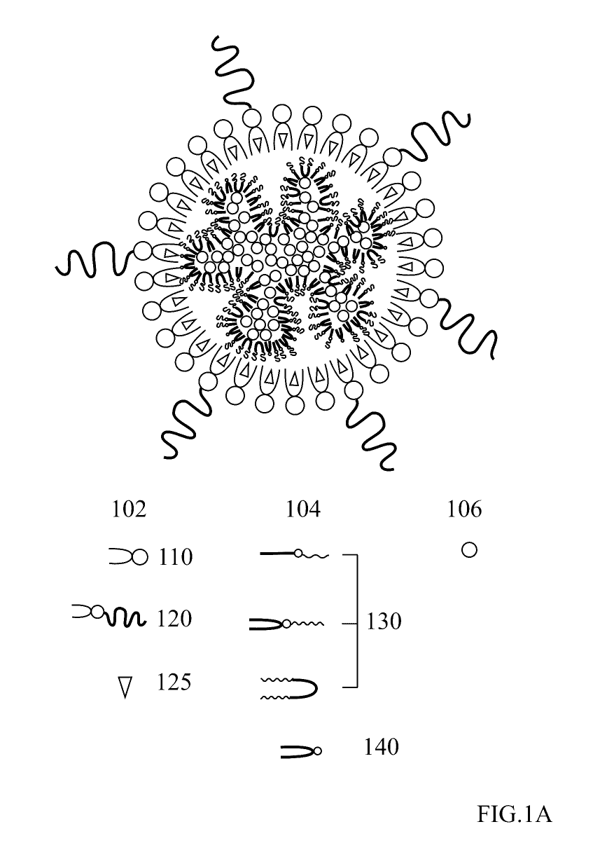

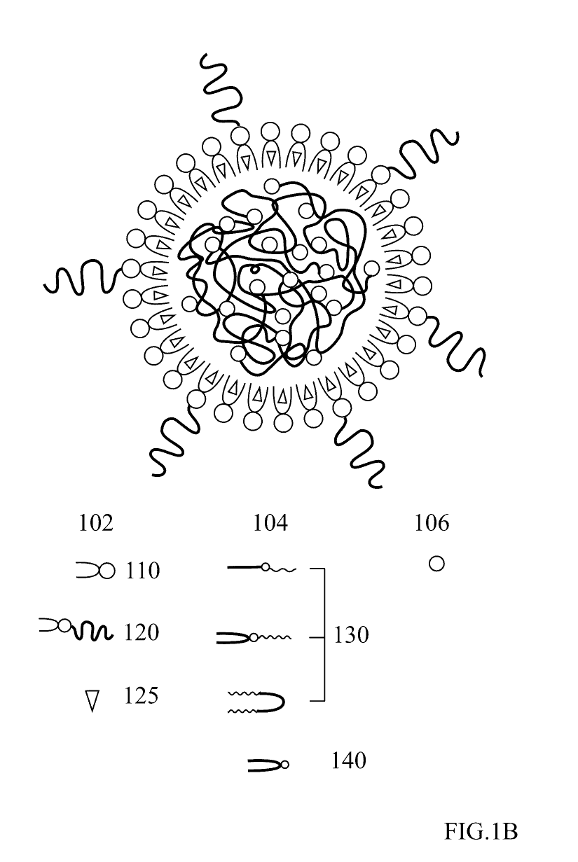

FIG. 1A and 1B illustrates a schematic diagram of a nanocarrier 100 according to the present disclosure. The nanocarrier 100 is composed of a micellar core 104 dispersed uniformly in a lipid shell 102. As shown in FIG. 1A, the lipid shell 102 comprises a phospholipid 110 and an amphiphilic lipid 120, and the phospholipid 110 is mixed with the amphiphilic lipid 120. The micellar core 104 is composed of an amphiphilic polymer 130 and a pharmaceutically or nutraceutically active ingredient 106. As shown in FIG. 1B, the lipid shell 102 comprises a phospholipid 110 mixed with pegylated phospholipid or cholesterol 125, or both. The micellar core 104 is composed of an amphiphilic polymer 130, phospholipid 140, and a pharmaceutically or nutraceutically active ingredient 150.

The phospholipid 110 of the lipid shell 102 is contributive to enclosing the micellar core 104, wherein hydrophobic molecules are evenly dispersed. According to an embodiment, the phospholipid 110 is lecithin, soybean lecithin, egg yolk lecithin or a synthetic phospholipid. The synthetic phospholipid is selected from phosphatidylcholines, phosphatidic acid, phosphatidylethanolamines, phosphatidylglycerols, phosphatidylserines, phosphatidylinositols, and a combination thereof.

The amphiphilic lipid 120 in the lipid shell 102 is selected from lipid-polyethyleneglycol conjugates, pegylated phospholipid, and a combination thereof.

The amphiphilic polymer 130 in the micellar core 104 is selected from poloxamers, poloxamines, TPGS, tween, or ethoxylated hydrogenated castor oil, pegylated phospholipid, and a combination thereof.

The micellar core 104 is composed of an amphiphilic polymer 130 and a phospholipid 140. The amphiphilic polymer 130 is DSPE-PEG2000 and the phospholipid is soybean lecithin.

According to an embodiment, a pharmaceutically or nutraceutically active ingredient 106 is docetaxel.

The above nanocarrier 100 is a core-shell nano-structure particle, and the diameter of the nanocarrier is in the range of about 100 nm to about 500 nm, preferably about 110 nm to about 200 nm, more preferably about 120 nm to about 150 nm.

The above nanocarrier 100 is prepared to have an encapsulating efficiency in the range of about 50 to about 100%, preferably about 80 to about 95%, more preferably about 90 nm to about 95%.

The above nanocarrier 100 is prepared to have a drug loading in the range of about 5 to about 15%, preferably about 6 to about 12%, more preferably about 8 to about 10%.

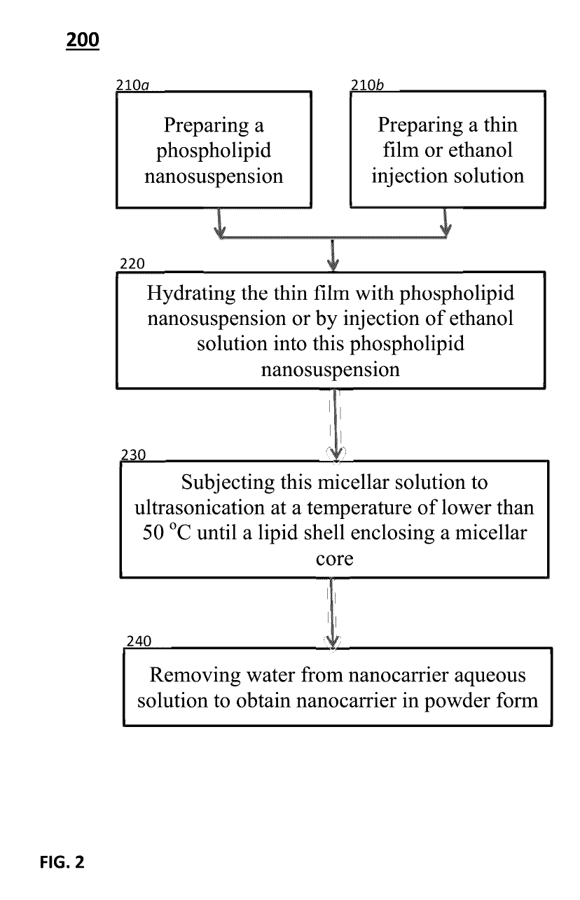

FIG. 2 illustrates a flow diagram of a method for preparing a nanocarrier. In the preparation method 200 as shown in FIG. 2, the first step is to prepare a phospholipid nanosuspension by subjecting phospholipid dispersion to ultrasonication 210a and a thin film or an ethanol solution 210b. Self-assembling micelles encapsulating a pharmaceutically or nutraceutically active ingredient are produced by hydrating the thin film composed of an amphiphilic polymer and a pharmaceutically or nutraceutically active ingredient with phospholipid nanosuspension or by injection of ethanol solution containing an amphiphilic polymer and a pharmaceutically or nutraceutically active ingredient into this phospholipid nanosuspension 220. The next step is to subject this micellar solution to ultrasonication at a temperature of lower than 50° C. until a lipid shell enclosing a micellar core composed of an amphiphilic polymer is formed, wherein a pharmaceutically or nutraceutically active ingredient is disposed 230. Optionally, the water is removed from nanocarrier aqueous solution to obtain nanocarrier in powder form 240. In step 210a, a thin film containing phospholipid and cholesterol is formed before subjection to ultrasonication to prepare phospholipid nanosuspension.

In step 210a, a phospholipid nanosuspension is prepared by dispersing a phospholipid in the aqueous solution and then subjecting it to probe ultrasonication. In an embodiment, the phospholipid is soybean lecithin and the amount used is at a weight ratio (w/w) to active ingredient of about 1.0 to 5.0, preferably about 2.0 to 3.0, prepared at a concentration of 1.0-5.0% (w/v). The aqueous solution is water.

In step 210b, an amphiphilic polymer and a pharmaceutically or nutraceutic ally active ingredient are dissolved in an organic solvent for forming the thin film. In an embodiment, the amphiphilic polymer is TPGS and the amount used is at a weight ratio (w/w) to active ingredient of about 1.0-10, preferably about 2.5-5.0. The pharmaceutically or nutraceutically active ingredient is docetaxel.

In step 210a and step 210b, the organic solvent is removed to obtain the thin film. In an embodiment, the method of removing the organic solvent is using a rotary vacuum evaporator.

In step 220, the thin film composed of an amphiphilic polymer and a pharmaceutically or nutraceutically active ingredient is hydrated with phospholipid nanosuspension to self-assembly form micelles encapsulating a pharmaceutically or nutraceutically active ingredient. In an embodiment, the amphiphilic polymer is DSPE-PEG2000 and the amount used is at a weight ratio (w/w) to active ingredient of about 1.0-10, preferably about 2.5-5.0. The pharmaceutically or nutraceutically active ingredient is docetaxel.

In step 230, the micellar solution is subjected to ultrasonication at a temperature of lower than 50° C. until a lipid shell enclosing a micellar core composed of an amphiphilic polymer and a pharmaceutically or nutraceutically active ingredient is formed. In one embodiment, cup ultrasonication is utilized at full power for 5 min while maintaining the temperature of solution at 25° C.

The ultrasonication method in the above step 210a and step 230 is using an ultrasonic processor implemented with cup and probe function.

In step 240, the method further comprises a step of removing water from the nanocarrier solution to obtain a nanocarrier in powder formulations by freeze-drying method. An appropriate amount of the anti-freezing agent is added to a solution having a nanocarrier. After being frozen at −80° C., it was transferred to a freeze dryer in an environment lower than −40° C. and 0.133 mbar for one day, thus obtaining the dry powdered nanocarrier.

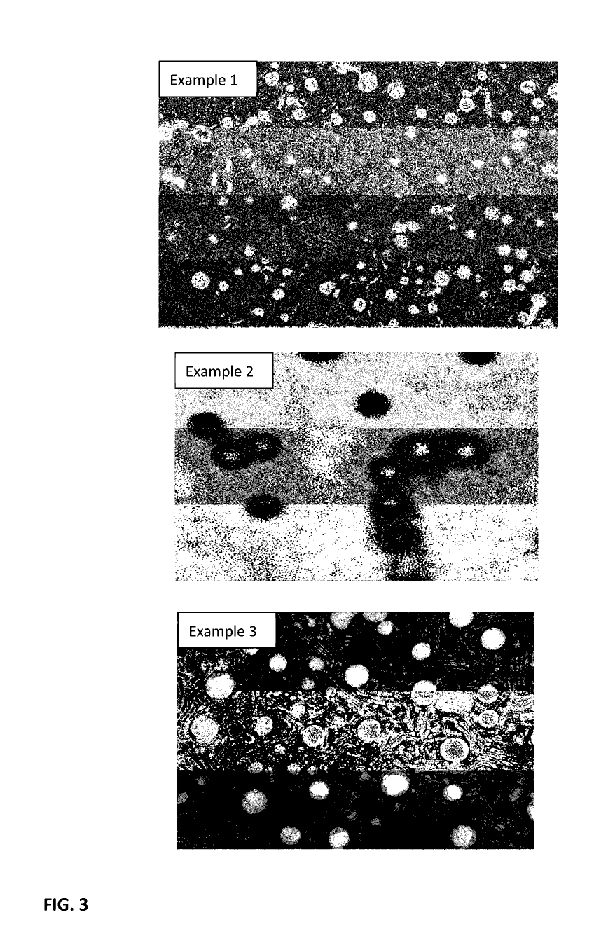

Three nanocarriers manufactured by three embodiments in the present disclosure are shown in FIG. 3A, 3B, and 3C, respectively. All show the drug is dispersed uniformly in the micellar core encapsulated with a lipid shell.

### Administration and Composition of Nanocarrier of the Invention

The nanocarrier containing load active agent or diagnostic agent can be administered to patients along with pharmaceutical excipients or diluents. Therefore, the invention also provides a composition comprising the nanocarriers of the invention and pharmaceutically or nutraceutic ally acceptable excipients or diluents. Non-limiting examples of suitable pharmaceutical excipients or diluents include starch, glucose, lactose, sucrose, gelatin, malt, rice, fluor, chalk, silica gel, magnesium carbonate, magnesium stearate, sodium stearate, glycerol monostearate, talc, sodium chloride, dried skim milk, glycerol, propylene, glycol, water, ethanol, buffered water, phosphate buffered saline and the like. These compositions can take the form of solutions, suspensions, tablets, pills, capsules, powders, sustained-release formulations and the like. It will be understood that the therapeutic dosage administered will be determined by the physician in the light of the relevant circumstances including the clinical condition to be treated and the chosen route of administration. Administration may be topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or oral administration.

The nanocarrier of the invention can be provided in lyophilized form for reconstitution, for instance, in isotonic, aqueous, or saline buffers for parental, subcutaneous, intradermal, intramuscular or intravenous administration. The subject composition of the invention may also be administered to the patient in need of a therapeutic agent by liquid preparations for orifice, e.g. oral, nasal, sublingual, administration such as suspensions, syrups or elixirs. The subject composition of the invention may also be prepared for oral administration such as capsules, tablets, pills, and the like, as well as chewable solid formulations. The subject composition of the invention may also be prepared as a cream for dermal administration such as liquid, viscous liquid, paste, or powder.

The presently disclosed compositions are designed to deliver an active or diagnostic agent, particularly in oral, intranasal, sublingual, intraduodenal, subcutaneous, buccal, intracolonic, rectal, vaginal, mucosal, pulmonary, transdermal, intradermal, parenteral, intravenous, intramuscular and ocular systems as well as to be able to traverse the blood-brain barrier. Administration of an active or diagnostic agent bound to hydrophobic-core carrier composition of the present invention results in an increased bioavailability of the active agent compared to administration of the active agent alone.

### EXAMPLES

### Example 1

### Preparation of Docetaxel Nanocarrier with DSPE-PEG2000 as Core and Soybean Lecithin as Lipid Shell

Anticancer drug docetaxel was used as the enclosed drug. Referring to the flow diagram of FIG. 2 for preparing a drug nanocarrier and the description of the above embodiments, 150 mg docetaxel and 375 mg DSPE-PEG2000 were first dissolved in organic solvent and the thin film was formed after evaporation of organic solvent. 1000 mg soybean lecithin was suspended in 25 mL deionized water and then subjected to ultrasonication for forming a lecithin nanosuspension. Then the lecithin nanosuspension was used to hydrate the thin film containing docetaxel and DSPE-PEG2000 and the mixture was subjected to ultrasonication at full power for at least 5 min while maintaining a constant temperature to obtain a solution having a nanocarrier. Uncapsulated drug was discarded by filtering this nanocarrier solution via 0.22 um membrane. An appropriate amount of the anti-freezing agent was added to the filtrate containing nanocarrier. After being frozen at −80° C., it was transferred to a freeze dryer in an environment lower than −40° C. and 0.133 mbar for one day, thus obtaining the dry powdered nanocarrier. After the dry powdered nanocarrier was reconstituted with deionized water, physical characteristics of nanocarrier were examined and the results were listed as follows: mean size, 164.6±2.51 nm; PI, 0.423±0.159; encapsulation efficiency, 93.06%; drug loading, 9.15%; stability at room temperature and 4° C. was >8 hr and >48 hr, respectively.

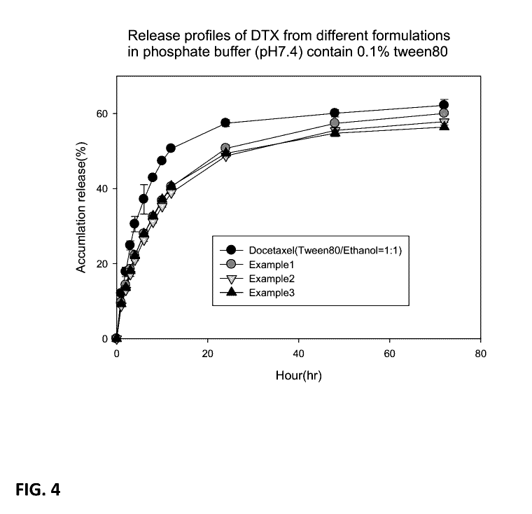

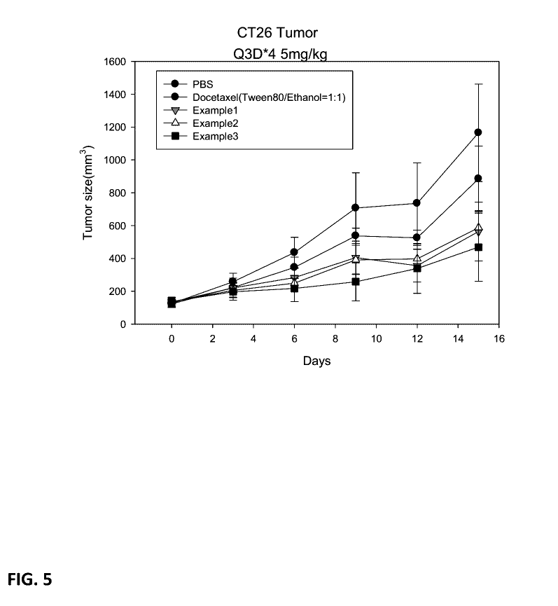

As shown in FIG. 3A, a docetaxel nanocarrier having a micellar core encapsulated with a lecithin shell was observed by transmission electron microscopy (TEM). FIG. 4 shows the drug release plot (Example 1) of doccetaxel from this nanocarrier at pH 7.4 environments according to one embodiment of this disclosure; FIG. 5 shows the volume change of CT-26 tumor xerograph illustrated by a mouse model treated with this docetaxel nanocarrier (Example 1) at a dosage regimen of 5 mg/kg q3dx4.

### Example 2

### Preparation of Docetaxel Nanocarrier with DSPE-PEG2000 as Core and Soybean Lecithin as Lipid Shell

Anticancer drug docetaxel was used as the enclosed drug. Referring to the flow diagram of FIG. 2 for preparing a drug nanocarrier and the description of the above embodiments, 150 mg docetaxel and 750 mg DSPE-PEG2000 were first dissolved in organic solvent and the thin film was formed after evaporation of organic solvent. 1000 mg soybean lecithin and 415 mg DSPE-PEG2000 were suspended in 25 mL deionized water and then subjected to ultrasonication for forming a lecithin/DSPE-PEG2000 nanosuspension. Then the lecithin nanosuspension was used to hydrate the thin film containing docetaxel and DSPE-PEG2000 and the mixture was subjected to ultrasonication at full power for at least 5 min while maintaining a constant temperature to obtain a solution having a nanocarrier. Uncapsulated drug was discarded by filtering this nanocarrier solution via 0.22 um membrane. An appropriate amount of the anti-freezing agent was added to the filtrate containing nanocarrier. After being frozen at −80° C., it was transferred to a freeze dryer in an environment lower than −40° C. and 0.133 mbar for one day, thus obtaining the dry powdered nanocarrier. After the dry powdered nanocarrier was reconstituted with deionized water, physical characteristics of nanocarrier were examined and the results were listed as follows: mean size, 154.0±1.45 nm; PI, 0.548±0.128; encapsulation efficiency, 97.3%; drug loading, 6.3%; stability at room temperature and 4° C. was >8 hr and >48 hr, respectively.

As shown in FIG. 3B, a docetaxel nanocarrier having a micellar core encapsulated with a lecithin/DSPE-PEG2000 shell was observed by transmission electron microscopy (TEM). FIG. 4 shows the drug release plot (Example 2) of doccetaxel from this nanocarrier at pH 7.4 environments according to one embodiment of this disclosure; FIG. 5 shows the volume change of CT-26 tumor xerograph illustrated by a mouse model treated with this docetaxel nanocarrier (Example 2) at a dosage regimen of 5 mg/kg q3dx4.

### Example 3

### Preparation of Docetaxel Nanocarrier with DSPE-PEG2000 as Core and Soybean Lecithin as Lipid Shell

Anticancer drug docetaxel was used as the enclosed drug. Referring to the flow diagram of FIG. 2 for preparing a drug nanocarrier and the description of the above embodiments, 75 mg docetaxel and 375 mg DSPE-PEG2000 were first dissolved in organic solvent and the thin film was formed after evaporation of organic solvent. A uniform mixture containing 500 mg soybean lecithin and 62.5 mg cholesterol was suspended in 12.5 mL deionized water and then subjected to ultrasonication for forming a lecithin/cholesterol nanosuspension. Then the lecithin nanosuspension was used to hydrate the thin film containing docetaxel and DSPE-PEG2000 and the mixture was subjected to ultrasonication at full power for at least 5 min while maintaining a constant temperature to obtain a solution having a nanocarrier. Uncapsulated drug was discarded by filtering this nanocarrier solution via 0.22 um membrane. An appropriate amount of the anti-freezing agent was added to the filtrate containing nanocarrier. After being frozen at −80° C., it was transferred to a freeze dryer in an environment lower than −40° C. and 0.133 mbar for one day, thus obtaining the dry powdered nanocarrier. After the dry powdered nanocarrier was reconstituted with deionized water, physical characteristics of nanocarrier were examined and the results were listed as follows: mean size, 112.7±2.43 nm; PI, 0.43±0.121; encapsulation efficiency, 93.3%; drug loading, 6.9%; stability at room temperature and 4° C. was >8 hr and >48 hr, respectively.

As shown in FIG. 3B, a docetaxel nanocarrier having a micellar core encapsulated with a lecithin/cholesterol shell was observed by transmission electron microscopy (TEM). As shown in FIG. 3A, a docetaxel nanocarrier having a micellar core encapsulated with a lecithin shell was observed by transmission electron microscopy (TEM). FIG. 4 shows the drug release plot (Example 3) of doccetaxel from this nanocarrier at pH 7.4 environments according to one embodiment of this disclosure; FIG. 5 shows the volume change of CT-26 tumor xerograph illustrated by a mouse model treated with this docetaxel nanocarrier (Example 3) at a dosage regimen of 5 mg/kg q3dx4.

### Example 4

### Preparation of Amphotericin B Nanocarrier with DSPE-PEG2000 as Core and Soybean Lecithin as Lipid Shell

Antifungal drug amphotericin B was used as the enclosed drug. Referring to the flow diagram of FIG. 2 for preparing a drug nanocarrier and the description of the above embodiments, 5 mg amphotericin B and 15 mg DSPE-PEG2000 were first dissolved in DMSO and the thin film was formed after freeze-drying. 20 mg soybean lecithin was suspended in 1.0 mL deionized water and then subjected to ultrasonication for forming a lecithin nanosuspension. Then the lecithin nanosuspension was used to hydrate the thin film containing amphotericin and DSPE-PEG2000 and the mixture was subjected to ultrasonication at full power for at least 5 min while maintaining a constant temperature to obtain a solution having a nanocarrier. Uncapsulated drug was discarded by filtering this nanocarrier solution via 0.22 um membrane. An appropriate amount of the anti-freezing agent was added to the filtrate containing nanocarrier. After being frozen at −80° C., it was transferred to a freeze dryer in an environment lower than −40° C. and 0.133 mbar for one day, thus obtaining the dry powdered nanocarrier. After the dry powdered nanocarrier was reconstituted with deionized water, physical characteristics of nanocarrier were examined and the results were listed as follows: mean size, 89.4±4.5 nm; PI, 0.679±0.244; encapsulation efficiency, 96.8%; drug loading, 12.5%.

### Example 5

### Preparation of Iirinotecan Nanocarrier with TPGS as Core and Soybean Lecithin as Lipid Shell

Anticancer drug irinotecan was used as the enclosed drug. Referring to the flow diagram of FIG. 2 for preparing a drug nanocarrier and the description of the above embodiments, 5 mg irinotecan and 10 mg TPGS were first dissolved in organic solvent and the thin film was formed after evaporation of the organic solvent. 30 mg soybean lecithin was suspended in 1.0 mL deionized water and then subjected to ultrasonication for forming a lecithin nanosuspension. Then the lecithin nanosuspension was used to hydrate the thin film containing irinotecan and TPGS and the mixture was subjected to ultrasonication at full power for at least 5 min while maintaining a constant temperature to obtain a solution having a nanocarrier. Uncapsulated drug was discarded by filtering this nanocarrier solution via 0.22 um membrane. An appropriate amount of the anti-freezing agent was added to the filtrate containing nanocarrier. After being frozen at −80° C., it was transferred to a freeze dryer in an environment lower than −40° C. and 0.133 mbar for one day, thus obtaining the dry powdered nanocarrier. After the dry powdered nanocarrier was reconstituted with deionized water, physical characteristics of nanocarrier were examined and the results were listed as follows: mean size, 107.6±2.0 nm; PI, 0.340±0.053; encapsulation efficiency, >90.0%; drug loading, 10.0%.

### Example 6

### Preparation of Curcumin Nanocarrier with Lecithin and Glycolate as Core and Soybean Lecithin as Lipid Shell

Nutraceutical drug curcumin was used as the enclosed drug. Referring to the flow diagram of FIG. 2 for preparing a drug nanocarrier and the description of the above embodiments, 5 mg curcumin, 3 mg lecithin, and 15 mg sodium glycolate were first dissolved in organic solvent and the thin film was formed after evaporation of the organic solvent. 20 mg soybean lecithin was suspended in 1.0 mL deionized water and then subjected to ultrasonication for forming a lecithin nanosuspension. Then the lecithin nanosuspension was used to hydrate the thin film containing curcumin/lecithin/sodium glycolate and the mixture was subjected to ultrasonication at full power for at least 5 min while maintaining a constant temperature to obtain a solution having a nanocarrier. Uncapsulated drug was discarded by filtering this nanocarrier solution via 0.22 um membrane. An appropriate amount of the anti-freezing agent was added to the filtrate containing nanocarrier. After being frozen at −80° C., it was transferred to a freeze dryer in an environment lower than −40° C. and 0.133 mbar for one day, thus obtaining the dry powdered nanocarrier. After the dry powdered nanocarrier was reconstituted with deionized water, physical characteristics of nanocarrier were examined and the results were listed as follows: mean size, 117.4±2.15 nm; PI, 0.900±0.037; encapsulation efficiency, >93.0%; drug loading, >8.0%.

### Example 7

### Preparation of Curcumin Nanocarrier with Lecithin and Pluronic P123 as Core and Soybean Lecithin as Lipid Shell

Nutraceutical drug curcumin was used as the enclosed drug. Referring to the flow diagram of FIG. 2 for preparing a drug nanocarrier and the description of the above embodiments, 5 mg curcumin, 2 mg lecithin, and 20 mg Pluronic P123 were first dissolved in organic solvent and the thin film was formed after evaporation of the organic solvent. 30 mg soybean lecithin was suspended in 1.0 mL deionized water and then subjected to ultrasonication for forming a lecithin nanosuspension. Then the lecithin nanosuspension was used to hydrate the thin film containing curcumin/lecithin/Pluronic P123 and the mixture was subjected to ultrasonication at full power for at least 5 min while maintaining a constant temperature to obtain a solution having a nanocarrier. Uncapsulated drug was discarded by filtering this nanocarrier solution via 0.22 um membrane. An appropriate amount of the anti-freezing agent was added to the filtrate containing nanocarrier. After being frozen at −80° C., it was transferred to a freeze dryer in an environment lower than −40° C. and 0.133 mbar for one day, thus obtaining the dry powdered nanocarrier. After the dry powdered nanocarrier was reconstituted with deionized water, physical characteristics of nanocarrier were examined and the results were listed as follows: mean size, 128.8±2.01 nm; PI, 0.553±0.060; encapsulation efficiency, >90.0%; drug loading, >8.0%.

### Example 8

### Preparation of Resveratrol Nanocarrier with Lecithin and Pluronic P123 as Core and Soybean Lecithin as Lipid Shell

Nutraceutical drug resveratrol was used as the enclosed drug. Referring to the flow diagram of FIG. 2 for preparing a drug nanocarrier and the description of the above embodiments, 5 mg resveratrol, 2 mg lecithin, and 20 mg Pluronic P123 were first dissolved in organic solvent and the thin film was formed after evaporation of the organic solvent. 20 mg soybean lecithin was suspended in 1.0 mL deionized water and then subjected to ultrasonication for forming a lecithin nanosuspension. Then the lecithin nanosuspension was used to hydrate the thin film containing resveratrol and TPGS and the mixture was subjected to ultrasonication at full power for at least 5 min while maintaining a constant temperature to obtain a solution having a nanocarrier. Uncapsulated drug was discarded by filtering this nanocarrier solution via 0.22 um membrane. An appropriate amount of the anti-freezing agent was added to the filtrate containing nanocarrier. After being frozen at −80° C., it was transferred to a freeze dryer in an environment lower than −40° C. and 0.133 mbar for one day, thus obtaining the dry powdered nanocarrier. After the dry powdered nanocarrier was reconstituted with deionized water, physical characteristics of nanocarrier were examined and the results were listed as follows: mean size, 101.8±1.02 nm; PI, 0.591±0.021; encapsulation efficiency, >95.0%; drug loading, >8.0%.

### Example 9

### Preparation of Honokiol/Magnolol Nanocarrier with Lecithin and Pluronic P123 as Core and Soybean Lecithin as Lipid Shell

Nutraceutical drug honokiol/magnolol was used as the enclosed drug. Referring to the flow diagram of FIG. 2 for preparing a drug nanocarrier and the description of the above embodiments, 6 mg honokiol/magnolol, 6 mg lecithin, and 60 mg Pluronics P123 were first dissolved in organic solvent and the thin film was formed after evaporation of the organic solvent. 30 mg soybean lecithin was suspended in 1.0 mL deionized water and then subjected to ultrasonication for forming a lecithin nanosuspension. Then the lecithin nanosuspension was used to hydrate the thin film containing honokiol/magnolol/lecithin/sodium glycolate and the mixture was subjected to ultrasonication at full power for at least 5 min while maintaining a constant temperature to obtain a solution having a nanocarrier. Uncapsulated drug was discarded by filtering this nanocarrier solution via 0.22 um membrane. An appropriate amount of the anti-freezing agent was added to the filtrate containing nanocarrier. After being frozen at −80° C., it was transferred to a freeze dryer in an environment lower than −40° C. and 0.133 mbar for one day, thus obtaining the dry powdered nanocarrier. After the dry powdered nanocarrier was reconstituted with deionized water, physical characteristics of nanocarrier were examined and the results were listed as follows: mean size, 147.1±3.11 nm; PI, 0.052±0.137; encapsulation efficiency, >95.0%; drug loading, >14.0%.

30 151.9±3.54 0.815±0.075

### Example 10

### Preparation of Honokiol/Magnolol Nanocarrier with Lecithin and Pluronic P123 as Core and Soybean Lecithin as Lipid Shell

Nutraceutical drug honokiol/magnolol was used as the enclosed drug. Referring to the flow diagram of FIG. 2 for preparing a drug nanocarrier and the description of the above embodiments, 1 mg honokiol/magnolol, 1 mg lecithin, and 10 mg Pluronic P123 were first dissolved in organic solvent and the thin film was formed after evaporation of the organic solvent. 30 mg soybean lecithin was suspended in 1.0 mL deionized water and then subjected to ultrasonication for forming a lecithin nanosuspension. Then the lecithin nanosuspension was used to hydrate the thin film containing honokiol/magnolol/lecithin/sodium glycolate and the mixture was subjected to ultrasonication at full power for at least 5 min while maintaining a constant temperature to obtain a solution having a nanocarrier. Uncapsulated drug was discarded by filtering this nanocarrier solution via 0.22 um membrane. An appropriate amount of the anti-freezing agent was added to the filtrate containing nanocarrier. After being frozen at −80° C., it was transferred to a freeze dryer in an environment lower than −40° C. and 0.133 mbar for one day, thus obtaining the dry powdered nanocarrier. After the dry powdered nanocarrier was reconstituted with deionized water, physical characteristics of nanocarrier were examined and the results were listed as follows: mean size, 151.9±3.54 nm; PI, 0.815±0.075; encapsulation efficiency, >85.0%; drug loading, >5.0%.

### Example 11

### Preparation of Quercetin Nanocarrier with TPGS as Core and Soybean Lecithin as Lipid Shell

Nutraceutical drug quercetin was used as the enclosed drug. Referring to the flow diagram of FIG. 2 for preparing a drug nanocarrier and the description of the above embodiments, 5 mg quercetin and 30 mg TPGS were first dissolved in organic solvent and the thin film was formed after evaporation of the organic solvent. 40 mg soybean lecithin was suspended in 1.0 mL deionized water and then subjected to ultrasonication for forming a lecithin nanosuspension. Then the lecithin nanosuspension was used to hydrate the thin film containing quercetin and TPGS and the mixture was subjected to ultrasonication at full power for at least 5 min while maintaining a constant temperature to obtain a solution having a nanocarrier. Uncapsulated drug was discarded by filtering this nanocarrier solution via 0.22 um membrane. An appropriate amount of the anti-freezing agent was added to the filtrate containing nanocarrier. After being frozen at −80° C., it was transferred to a freeze dryer in an environment lower than −40° C. and 0.133 mbar for one day, thus obtaining the dry powdered nanocarrier. After the dry powdered nanocarrier was reconstituted with deionized water, physical characteristics of nanocarrier were examined and the results were listed as follows: mean size, 107.6±2.0 nm; PI, 0.340±0.053; encapsulation efficiency, >90.0%; drug loading, >5.0%.

### Example 12

### Preparation of Docetaxel Nanocarrier with PLGA as Core and Lecithin and DSPE-PEG2000 as Lipid Shell

Anticancer drug docetaxel was used as the enclosed drug. Referring to the flow diagram of FIG. 2 for preparing a drug nanocarrier and the description of the above embodiments, 3.75 mg docetaxel and 25 mg PLGA were first dissolved in water miscible organic solvent to form injection solution. 5 mg soybean lecithin and 5 mg DSPE-PEG2000 were suspended in 10.0 mL deionized water and then subjected to ultrasonication for forming a lecithin nanosuspension. Then the docetaxel/PLGA solution was injected into the lecithin nanosuspension and the mixture was subjected to ultrasonication at full power for at least 5 min while maintaining a constant temperature to obtain a solution having a nanocarrier. Uncapsulated drug was discarded by filtering this nanocarrier solution via 0.22 um membrane. An appropriate amount of the anti-freezing agent was added to the filtrate containing nanocarrier. After being frozen at −80° C., it was transferred to a freeze dryer in an environment lower than −40° C. and 0.133 mbar for one day, thus obtaining the dry powdered nanocarrier. After the dry powdered nanocarrier was reconstituted with deionized water, physical characteristics of nanocarrier were examined and the results were listed as follows: mean size, 122.5±0.93 nm; PI, 0.178±0.072; encapsulation efficiency, >50.0%; drug loading, >5.0%.

The above embodiments/examples in the present disclosure use the properties of micelles to prepare a nanocarrier core, and the lipid shell structure can be applied to encapsulate such a micellar core forming a nanocarrier. By the hydration of lecithin nanosuspension, the amphiphilic polymers with or without lecithin self-assemble to form the micellar core encapsulated with a lipid shell to form a nanocarrier. All materials employed in this nanocarrier have the advantages of being less expensive and having high biocompatibility and degradability. These features mean the micelles enclose each kind of drug effectively, help to increase the payload efficiency, and decrease drug leakage.

The micellar core in the nanocarrier encapsulated with a lipid shell can reduce the problems of high drug leakage and instability resulting when the drug is only entrapped in the matrix of amphiphilic polymer. Furthermore, the particle size of the nanocarrier is in a nano range with a lipid surface, which makes it highly permeable through various biological membrane barriers, such that it is administrable not only intravenously, but also via various routes including subcutaneously, dermally, orally, mucously, sublingually, and ocularly, to enable new application platforms for drug delivery in the future.

All the features disclosed in this specification (including any accompanying claims, abstract, and drawings) may be replaced by alternative features serving the same, equivalent or similar purpose, unless expressly stated otherwise. Thus, each feature disclosed is one example only of a generic series of equivalent or similar features.

What is claimed is:

1. A nanocarrier comprising a lipid shell enclosing a micellar core encapsulating an active agent or a diagnostic agent, wherein the lipid shell comprises one or more amphiphilic lipids, and the micellar core comprises one or more amphiphilic polymers wherein the core optionally comprises an emulsifier.

2. The nanocarrier of claim 1, wherein the diameter of the nanocarrier is in the range of about 50 nm to about 500 nm.

3. The nanocarrier of claim 1, which has an encapsulating efficiency in the range of about 50 to about 100%.

4. The nanocarrier of claim 1, wherein the amphiphilic lipid is selected from the group consisting of lipid-polyethyleneglycol conjugate, phospholipid, or cholesterol or a combination thereof.

5. The nanocarrier of claim 4, wherein the phospholipid is lecithin, soybean lecithin, egg yolk lecithin, a synthetic phospholipid or a pegylated phospholipid.

6. The nanocarrier of claim 5, wherein the synthetic phospholipid is phosphatidylcholine, phosphatidic acid, phosphatidylethanolamine, phosphatidylglycerol, phosphatidylserine, phosphatidylinositol, or a combination thereof. In a further embodiment, the amphiphilic lipid is a lipid-polyethyleneglycol conjugate, pegylated phospholipid, or a combination thereof.

7. The nanocarrier of claim 1, wherein the lipid shell comprises a phospholipid and another amphiphilic lipid selected from pegylated phospholipid and cholesterol.

8. The nanocarrier of claim 1, wherein the lipid shell comprises a lipid-polyethyleneglycol conjugates, pegylated phospholipid, or a combination thereof.

9. The nanocarrier of claim 1, wherein the lipid shell comprises a phospholipid and a pegylated phospholipid or cholesterol.

10. The nanocarrier of claim 1, wherein the amphiphilic polymer is selected from the group consisting of phospholipid, poloxamer, poloxamine, TPGS, tween, or ethoxylated hydrogenated castor oil, pegylated phospholipid, PLGA, PLA, PGA, and a combination thereof.

11. The nanocarrier of claim 10, wherein the phospholipid is lecithin.

12. The nanocarrier of claim 1, wherein the emulsifier is selected from the group consisting of sodium glycocholate, sodium taurocholate and sodium taurodeoxycholate.

13. The nanocarrier of claim 1, wherein the micellar core comprises a phospholipid and another amphiphilic polymer selected from DSPE-PEG2000, PLGA, Pluronic P123 and a combination thereof.

14. The nanocarrier of claim 1, wherein the micellar core comprises a combination of lecithin and Pluronic P123 or a combination of lecithin and sodium glycolate.

15. The nanocarrier of claim 1, wherein the active agent is an anti-cancer drug, an antimicrobial drug or a nutraceutical agent and the diagnostic agent is an imaging agent, an enzyme, a fluorescent substance, a luminescent substance or a paramagnetic molecule.

16. The nanocarrier of claim 1, wherein the loading of the active agent in the nanocarrier is in the range of about 5% to about 15%.

17. A method of preparing a nanocarrier with higher bioactive or diagnostic agent loading, comprising (i) preparing a nanosuspension comprising one or more amphiphilic lipids by subjecting the one or more amphiphilic lipids to ultrasonication; (iia) preparing a thin film comprising a mixture of one or more amphiphilic polymers wherein the mixture optionally comprises an emulsifier and an active agent or a diagnostic agent by dissolving the mixture in an organic solvent and then removing the organic solvent or (iib) dissolving the mixture in an organic solvent to form an organic solution; (iii) hydrating the thin film of (iia) with the nanosuspension or injecting the organic solution of (iib) into the nanosuspension to form a solution containing self-assembling micelles encapsulating the active agent or diagnostic agent; and (iv) subjecting the micellar solution to ultrasonication at a temperature of lower than 50° C. until the amphiphilic lipid forms a lipid shell and then encloses micelles as a core.

18. The method of claim 17, which further comprises further comprises a step of removing water from the nanocarrier aqueous solution to obtain nanocarrier in powder form.

19. The method of claim 17, wherein the nanosuspension contains an amphiphilic lipid having a weight ratio (w/w) to an active agent or a diagnostic agent about 1.0 to 5.0 prepared at a concentration of 1.0-5.0% (w/v).

20. The method of claim 17, wherein the amount of the amphiphilic polymer in the thin film or in the organic solution is at a weight ratio (w/w) to active ingredient about 1.0-10.
